(19) United States
(12) Patent Application Publication
(10) Pub. No.: US 2013/0103000 A1
(43) Pub. Date: Apr. 25, 2013

(54) CATHETER ASSEMBLY

(71) Applicants: Michael A. Vogelbaum, Moreland Hills, OH (US); Ji-Feng Chen, Lakewood, OH (US)

(72) Inventors: Michael A. Vogelbaum, Moreland Hills, OH (US); Ji-Feng Chen, Lakewood, OH (US)

(73) Assignee: THE CLEVELAND CLINIC FOUNDATION, Cleveland, OH (US)

(21) Appl. No.: 13/606,187

(22) Filed: Sep. 7, 2012

Related U.S. Application Data
(60) Provisional application No. 61/532,591, filed on Sep. 9, 2011.

(51) Int. Cl. A61M 25/00; A61M 25/01

(52) U.S. Cl. CPC A61M 25/0021; A61M 25/0102
USPC 604/528; 604/523

(57) ABSTRACT

A catheter assembly comprises a catheter having a wall at least partially defining a lumen. The lumen communicates with an opening in an exterior catheter surface at a distal end of the catheter. A lengthwise portion of the wall is made of a resilient material and is longitudinally extensible. The lengthwise portion of the wall has a diameter that is reduced when the lengthwise portion of the wall is longitudinally extended. The diameter of the lengthwise portion of the wall increases from a reduced condition when the lengthwise portion of the wall resiliently returns from a longitudinally extended condition.

### RELATED APPLICATIONS

This application claims priority from U.S. Provisional Application No. 61/532,591, filed 9 Sep. 2011, and U.S. patent application Ser. No. 13/044,963, filed 10 Mar. 2011, the subject matter of which is incorporated herein by reference in its entirety.

### FIELD OF THE INVENTION

The present invention relates to a catheter assembly that is longitudinally extensible and, more particularly, to a catheter assembly in which a catheter wall is reduced in diameter when a portion of the wall is longitudinally extended.

### BACKGROUND OF THE INVENTION

Convection enhanced delivery (“CED”) of a bioactive agent involves introducing a fluid containing the bioactive agent into a patient's tissue under pressure so that the fluid moves through the tissue via bulk flow. Implementing CED generally involves inserting catheters into the tissue to be treated, such as cerebral tissue. To reduce the risk of hemorrhage and/or trauma to the tissue, it is desirable for such a catheter to have a small outside diameter. It is also desirable to prevent backflow of fluid along the outside of the catheter.

### SUMMARY OF THE INVENTION

The present invention is directed to a catheter assembly that is longitudinally extensible and, more particularly, to a catheter assembly in which a catheter wall is reduced in diameter when a portion of the wall is longitudinally extended.

In accordance with an embodiment of the present invention, a catheter assembly comprises a catheter having a wall at least partially defining a lumen. The lumen communicates with an opening in an exterior catheter surface at a distal end of the catheter. A lengthwise portion of the wall is made of a resilient material and is longitudinally extensible. The lengthwise portion of the wall has a diameter that is reduced when the lengthwise portion of the wall is longitudinally extended. The diameter of the lengthwise portion of the wall increases from a reduced condition when the lengthwise portion of the wall resiliently returns from a longitudinally extended condition.

### BRIEF DESCRIPTION OF THE DRAWINGS

The foregoing and other features and advantages of the present invention will become apparent to one skilled in the art upon consideration of the following description of the invention and the accompanying drawings, in which:

FIG. 1 is a sectional view of an embodiment of a catheter assembly in accordance with the present invention in a non-extended condition;

FIG. 2 is an enlarged sectional view of a distal portion of the catheter assembly of FIG. 1; and

FIG. 3 is a sectional view of the catheter assembly of FIG. 1 in a longitudinally extended condition.

### DETAILED DESCRIPTION

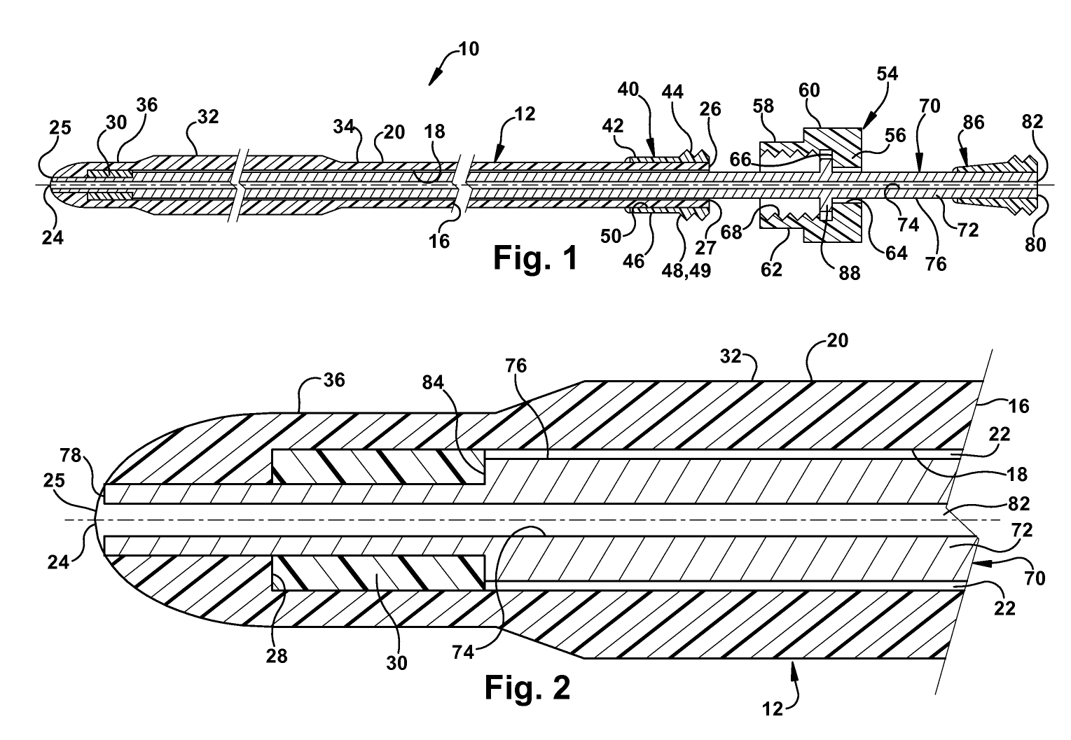

FIGS. 1 and 2 illustrate a catheter assembly 10, in accordance with an example of the present invention. The catheter assembly 10 includes a catheter 12. The catheter 12 is made of a flexible and resilient biocompatible material, such as a medical grade silicone elastomer, and includes a longitudinally extending, tubular wall 16. The tubular wall 16 includes a radially inner surface 18 and a radially outer surface 20. Both the inner surface 18 and the outer surface 20 extend substantially the entire length of the catheter 12 from a distal end 24 of the catheter to an opposite proximal end 26 of the catheter.

The inner surface 18 of the wall 16 defines a central lumen 22 (FIG. 2) that also extends substantially the entire length of the catheter 12. The central lumen 22 is open at the distal end 24 of the catheter 12. In other words, the central lumen 22 communicates with a port or opening 25 in the exterior surface of the catheter 12 at its distal end 24. The central lumen 22 is also open at the opposite, proximal end 26 of the catheter 12. As it does at the distal end 24 of the catheter 12, the central lumen 22 communicates with a port or opening 27 in the exterior surface of the catheter at its proximal end 26. Because of the foregoing construction, fluid may be introduced into the central lumen 22 through the opening 27 in the proximal end 26 of the catheter 12, flow along the central lumen 22, and then flow out the open distal end 24 of the catheter through the opening 25.

The inner surface 18 of the wall 16 has a substantially constant diameter from the proximal end 26 of the catheter 12 to a location adjacent to but spaced from, the distal end 24. At this location, the diameter of the inner surface 18 is reduced, thereby forming a radially extending surface 28 in the wall 16. The surface 28 is thus a portion of the inner surface 18 oriented in a radial direction transverse to the length of the catheter 12. From the radially extending surface 28 to the distal end 24 of the catheter 12, the inner surface 18 of the wall 16 has a substantially constant diameter that is smaller than the diameter of the inner surface between the radially extending surface and the proximal end 26 of the catheter.

A tubular stopper element 30 is disposed in the central lumen 22 of the catheter 12 adjacent the radially extending surface 28. The outer diameter of the stopper element 30 is approximately the same as the diameter of the inner surface 18 of the wall 16 between the radially extending surface 28 and the proximal end 26 of the catheter 12. As a consequence, the stopper element 30 is supported on, and positioned in the central lumen 22 by, the radially extending surface 28. The stopper element 30, which may be formed of medical grade tubing, is secured to the inner surface 18 of the wall 16 by a biocompatible adhesive (not shown).

The outer surface 20 of the wall 16 is separated from the inner surface 18 by a first distance in a first portion 32 of the length of the wall. The first portion 32 of the length of the wall 16 extends from a location adjacent to a proximal end of the stopper element 30 toward the proximal end 26 of the catheter 12. The outer surface 20 of the wall 16 is separated from the inner surface 18 by a second distance, which is smaller than the first distance, in a second portion 34 of the length of the wall 16. The second portion 34 of the length of the wall 16 extends from the first portion 32 to the proximal end 26 of the catheter 12. As a consequence of the difference between the first and second distances, the wall 16 has a greater thickness in the first portion 32 of its length than in the second portion 34. The greater thickness of the first portion 32 of the wall 16 may be achieved, for example, by molding the wall in a single piece with a greater thickness in the first portion or by molding the wall with a constant thickness equal to the desired thickness of the second portion 34 of the wall and laminating one or more additional layers of material onto the wall to increase the wall thickness in the first portion 32 of the wall.

In a third portion 36 of the wall 16, which extends from a location adjacent to the proximal end of the stopper element 30 toward the distal end 24 of the catheter 12, the outer surface 20 of the wall 16 is separated from the inner surface 18 by a third distance. The third distance is smaller than the first distance and may be the same as the second distance. As a consequence of the difference between the first and third distances, the wall 16 has a greater thickness in the first portion 32 of its length than in the third portion 36. The diameter of the outer surface 20 of the wall 16 in the third portion 36 of the length of the wall 16 may be equal to or less than the outer diameter of the wall in the first portion 32 of the wall, measured when the first portion of the wall is extended or distended as described below.

Adjacent its proximal end 26, the catheter 12 is received in a tubular connector 40, such as a modified female Luer lock connector. The connector 40 has a head portion 42 and an opposite threaded portion 44. The head portion 42 of the connector 40 has an outer surface 46 formed with raised, longitudinally extending ridges to facilitate manipulation of the connector. The threaded portion 44 of the connector 40 has an outer surface 48 in which a screw thread 49 is formed. An inner surface 50 of the connector 40 extends through both the head portion 42 and the threaded portion 44 of the connector and defines a central passage in the connector. The portion of the catheter 12 adjacent its proximal end 26 is received in the central passage of the connector 40 with the threaded portion 44 of the connector adjacent the open proximal end 26 of the catheter and with the head portion 42 of the connector closer to the distal end 24 of the catheter 12. A biocompatible adhesive (not shown) fixes the inner surface 50 of the connector 40 to the outer surface 20 of the wall 16 of the catheter 12.

In use, the threaded portion 44 of the connector 40 is received in a threaded nut 54. The threaded nut 54 has an enlarged head portion 56 and an opposite threaded portion 58. The head portion 56 of the threaded nut 54 has an outer surface 60 formed in a rounded hexagonal shape with raised, longitudinally extending ridges at the corners of the hexagonal shape to facilitate manipulation of the threaded nut. The threaded portion 58 of the threaded nut 54 has a cylindrical outer surface 62. An inner surface 64 of the threaded nut 54 extends through both the head portion 56 and the threaded portion 58 of the threaded nut and defines a central passage in the threaded nut. The inner surface 64 includes a radial step 66 such that the central passage of the threaded nut 54 has a larger diameter adjacent the threaded portion 58 of the threaded nut and a smaller diameter adjacent the head portion 56 of the threaded nut. A screw thread 68 is formed in the inner surface 64 of the threaded nut 54 in the threaded portion 58 of the threaded nut.

The threaded portion 44 of the connector 40 is received in the threaded portion 58 of the threaded nut 54 with the screw thread 49 in the outer surface 48 of the threaded portion 44 engaging the screw thread 68 formed in the inner surface 64 of the threaded nut. An O-ring or annular washer (not shown), which may be formed of polytetrafluoroethylene (“PTFE”), for example, may be received against the inner surface 64 of the threaded nut 54 in the larger diameter portion of the central passage of the threaded nut between the end of the threaded portion 44 of the connector 40 and the head portion 56 of the threaded nut.

When the catheter assembly 10 is to be inserted into tissue, such as cerebral tissue, of a patient, a stylet 70 is inserted into the catheter 12. The stylet 70 is hollow and elongated and is formed of a relatively strong and rigid material, such as stainless steel. The stylet includes a longitudinally extending, tubular wall 72. The tubular wall 72 includes a radially inner surface 74 and a radially outer surface 76. Both the inner surface 74 and the outer surface 76 extend substantially the entire length of the stylet 70 from a distal end 78 (best shown in FIG. 2) of the stylet to an opposite proximal end 80 of the stylet. Although both FIG. 1 and FIG. 2 show a space between the inner surface 18 of the wall 16 of the catheter 12 and most of the outer surface 76 of the stylet 70, such a space need not be as large as illustrated and is not required at all. The diameter of the inner surface 18 and the diameter of the outer surface 76 may, therefore, be the same and typically would be substantially the same, except adjacent the stopper element 30.

The inner surface 74 of the wall 72 defines a stylet lumen 82 that also extends substantially the entire length of the stylet 70. The stylet lumen 82 is open at the distal end 78 of the stylet 70 and at the opposite, proximal end 80 of the stylet. As a result of the foregoing construction, fluid may flow along the stylet lumen 82 and then out the open distal end 78 of the stylet 70.

The inner surface 74 of the wall 72 has a substantially constant diameter from the distal end 78 of the stylet 70 to the proximal end 80. The outer surface 76 of the stylet 70, on the other hand, has two different diameters, as best seen in FIG. 2. Specifically, the outer surface 76 of the stylet 70 has first, larger diameter from the proximal end 80 of the stylet to a location adjacent to, but spaced from, the distal end 78. At this location, the diameter of the outer surface 76 is reduced, thereby forming a radially extending surface 84 (FIG. 2) in the wall 72. The surface 84 of the stylet 70 is thus a portion of the outer surface 76 oriented in a radial direction transverse to the length of the stylet. From the radially extending surface 84 to the distal end 78 of the stylet 70, the outer surface 76 of the wall 72 has a substantially constant second diameter that is smaller than the first diameter. When the stylet 70 is inserted into the catheter 12, the radially extending surface 84 is engageable with the tubular stopper element 30, as described below.

At the proximal end 80 of the stylet 70, a connector 86, such as a male Luer lock connector, is secured to the stylet. Adjacent the proximal end 80 of the stylet 70, but longitudinally spaced from the connector 86, an annular stroke limiter 88 encircles and is fixed to the stylet. The stylet 70 is first inserted into the central passage of the threaded nut 54, then into the central passage of the connector 40, and finally into the central lumen 22 of the catheter 12 until the radially extending surface 84 contacts the proximal end of the stopper element 30 secured to the inner surface 18 of the wall 16 of the catheter. The radially extending surface 84 of the stylet 70 is thus positioned to transmit to the radially extending surface 28 of the wall 16 of the catheter 12, through the stopper element 30, any pressure applied to the stylet.

After the radially extending surface 84 of the stylet 70 contacts the stopper element 30, the stylet continues to be pushed into the catheter 12 and against the stopper element until the stroke limiter 88 contacts the proximal end 26 of the catheter and the adjacent end of the threaded portion 44 of the connector 40. The continued pressure of the stylet 70 against the stopper element 30 causes the resilient material of which the wall 16 is made to stretch and thereby causes the wall 16 to extend or distend axially or lengthwise into a longitudinally extended condition. This stretching of the wall 16 occurs primarily in the thickened first portion 32 of the wall because the stopper element 30 is bonded to the inner surface 18 of the wall and effectively transfers the force applied by the stylet to the wall 16 adjacent the end of the first portion closest to the distal end 24 of the catheter 12.

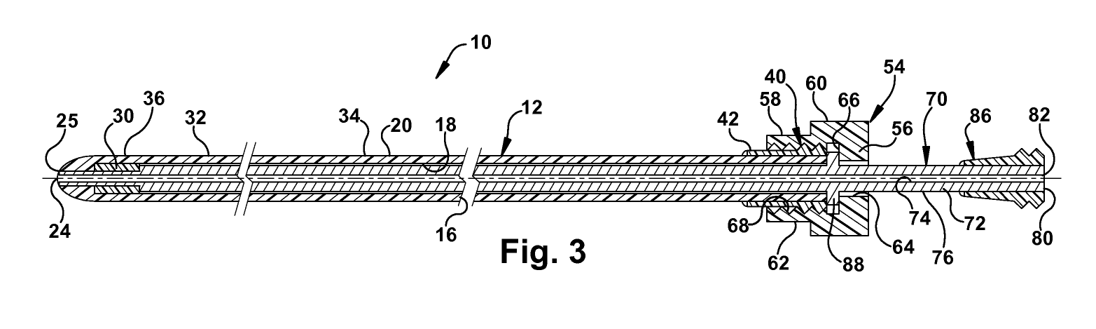

As can be seen in FIG. 3, longitudinal stretching of the wall 16 causes the outer diameter of the wall to decrease or be reduced. The outer diameter of the first portion 32 of the wall 16 may be reduced to any desired extent, such as less than or equal to the outer diameter of the second portion 34 of the wall. When the stylet 70 reaches the end of its stroke, as determined by contact between the stroke limiter 88 and the proximal end 26 of the catheter and the adjacent end of the threaded portion 44 of the connector 40, the stylet may be secured in place to facilitate coordinated manipulation of the stylet and the catheter assembly 10. Specifically, the threaded portion 44 of the connector 40 is received in the threaded portion 58 of the threaded nut 54 with the screw thread 49 in the outer surface 48 of the threaded portion 44 engaging the screw thread 66 formed in the inner surface 64 of the threaded nut.

When the threaded nut 54 is screwed onto the connector 40, the stroke limiter 88 of the stylet 70 is trapped between the threaded portion 44 of the connector and head portion 56 of the threaded nut. The stylet 70 and the catheter 12 then tend to move more consistently as a single unit and can be manipulated more easily and accurately. In particular, the stylet 70 can then be used to insert the extended catheter 12 into the tissue of a patient. Because the outer diameter of the catheter 12 has been reduced due to the lengthwise extension or distension of the catheter, the opening formed in the patient's tissue is smaller than it would be otherwise.

When the distal end 24 of the catheter 12 is appropriately positioned in a patient's tissue, the stylet 70 is held so as to maintain the distal end of the catheter in position. The threaded nut 54 may then be unscrewed from the connector 40 so that the stroke limiter 88 of the stylet 70 is no longer trapped between the threaded portion 44 of the connector and head portion 56 of the threaded nut. With the stylet 70 held in position and the stroke limiter 88 no longer trapped between the connector 40 and the threaded nut 54, respectively, the resilience of the extended catheter 12 pulls the proximal end 26 of the catheter along the stylet toward the distal end 24 of the catheter. The catheter 12 thus returns resiliently to its initial, non-extended length while the distal end 24 of the catheter remains in position.

As the catheter 12 resiliently returns to its initial, non-extended length and the wall 16 of the catheter likewise resiliently returns from its longitudinally extended condition to its initial, non-extended length, the outer diameter of the wall, particularly the first portion 32, increases from its reduced condition back to its original dimension. The increase in the outer diameter of the wall 16 of the catheter 12 causes the outer surface 20 of the wall 16 to press tightly against adjacent surfaces of the patient's tissue. The resulting close fit between the outer surface 20 of the wall 16 and the adjacent surfaces of the patient's tissue helps to prevent fluid introduced into the tissue by the catheter 12 from flowing back along the outer surface of the wall toward the proximal end 26 of the catheter.

With the catheter 12 of the catheter assembly 10 appropriately positioned in the patient's tissue, therapeutic treatment of the tissue with a bioactive material can begin. To introduce the bioactive material, the stylet 70 is withdrawn entirely from the central lumen 22 of the catheter 12 and from the connector 40. Before the stylet 70 is withdrawn from the catheter 12, however, the connector 86 at the proximal end 80 of the stylet is connected to a source of fluid, such as a liquid containing the bioactive material with which the patient's tissue is to be treated. As the stylet 70 is slowly being withdrawn from the catheter 12, the fluid is simultaneously being introduced into the stylet lumen 82. The simultaneous withdrawal of the stylet 70 from the central lumen 22 of the catheter and introduction of the fluid into the stylet lumen 82 permits the fluid to flow through the stylet lumen and out the open distal end 78 of the stylet to occupy the space in the central lumen being vacated by the stylet. This fluid flow helps to prevent other material, such as air, the patient's tissue, or fluid in the patient's tissue, from being drawn into the central lumen 22 of the catheter 12.

After the stylet 70 is withdrawn entirely from the central lumen 22 of the catheter 12, the threaded portion 44 of the connector 40 fixed to the catheter 12 is connected with a connector (not shown) and the distal end of a length of tubing (not shown). A proximal end (not shown) of the tubing is attached to a device (not shown), such as a pump, for delivering a fluid, such as a liquid. The fluid contains a bioactive material, such as a pharmaceutical material, and is delivered from the tubing into the central lumen 22 of the catheter 12. The fluid flows along the central lumen 22 of the catheter 12 until it reaches the open end of the distal end 24 of the catheter and is thereby introduced into the patient's tissue. When the patient's treatment is completed, the catheter assembly 10 may be removed by reintroducing the stylet 70 into the catheter assembly to extend or distend the catheter 12 and then withdrawing the catheter assembly from the patient's tissue.

Although fluid may be introduced into the stylet lumen 82 during withdrawal of the stylet 70 from the central lumen 22 of the catheter 12, fluid is always introduced into the patient's tissue through the opening 25 in the exterior surface of the catheter at its distal end 24. The catheter 12 does not include any other opening for introducing fluid into the patient's tissue. In addition, the catheter assembly 10 includes only a single catheter, namely, the catheter 12, and does not include any catheter other than the catheter 12, including, without limitation any catheter in the wall 16 of the catheter 12 or any catheter in the central lumen 22 of the catheter 12. More specifically, the catheter assembly 10 does not include any catheter that projects from the catheter 12 to penetrate the patient's tissue to introduce fluid into the patient's tissue.

As described above, the catheter 12 seals against a patient's tissue by having a thicker first portion 32 of the length of the wall 16 of the catheter and by extending or stretching the first portion of the wall to reduce its outer diameter when inserting the catheter and then allowing the first portion to return to its larger diameter after the catheter is properly positioned. This effect may be enhanced by forming the first portion 32 of the length of the wall 16 of the catheter 12 from a first material and forming the second and/or third portions 34 and 36 of the wall from another material or materials having different properties than the first material.

For example, the first portion 32 of the wall 16 may be formed of an elastomeric material that has a relatively low modulus of elasticity and a relatively low durometer and, therefore, is relatively extensible. The elastomeric material in the second portion 34 and/or third portion 36 of the length of the wall 16 may be formed of an elastomeric material that has a relatively high modulus of elasticity and a relatively high durometer and, therefore, is relatively inextensible. Similarly, the tubular stopper element 30 is disposed in the central lumen 22 of the catheter 12 may be formed of an elastomeric material that a relatively high modulus of elasticity and a relatively high durometer and, therefore, may be relatively inextensible. If the stopper element 30 is relatively inextensible, the stopper element will restrict or limit the extension or distension of the wall 16 adjacent to the stopper element, even though the wall adjoining the stopper element is formed of relatively extensible material, because the stopper element is secured to the wall.

While the catheter 12 has been described as being introduced into a patient's tissue and then later removed from the patient's tissue, the catheter may be fabricated of a material or materials that can be absorbed by the tissue, thereby reducing or eliminating the requirement physically to remove the catheters from the patient's tissue. Also, while the use of biocompatible adhesive materials has been described above to secure the connector 40 to the catheter 12, as well as to secure or attach together other components of the catheter assembly 10, other suitable attachment or fixation mechanisms, such as radio frequency welding and molded interlocking pins or other interlocking structural features, may be used where appropriate.

It will be appreciated that the catheter assembly 10 be used to treat both neoplastic and non-neoplastic disorders. Bioactive materials introduced into a patient's tissue using any of the catheter assembly 10 may include, for example, chemotherapeutic materials, viruses, proteins, radiologic materials, growth factors, peptides, and non-radioactive tracer molecules. The catheter assembly 10 may be used in a variety of patient tissues, including, for example, brain tissue, spinal cord tissue, and tissue of any organ.

From the above description of the invention, those skilled in the art will perceive improvements, changes and modifications. Such improvements, changes, and/or modifications within the skill of the art are intended to be covered by the appended claims.

1. A catheter assembly comprising a catheter having a wall at least partially defining a lumen, the lumen communicating with an opening in an exterior catheter surface at a distal end of the catheter, the wall of the catheter having an inner surface and an outer surface, the inner and outer surfaces of the wall being spaced apart (a) a first distance in a first lengthwise portion of the wall and (b) a second distance in a second lengthwise portion of the wall, the first distance being larger than the second distance, the first lengthwise portion of the wall being made of a resilient material and being longitudinally extensible, the first lengthwise portion of the wall having a diameter that is reduced when the first lengthwise portion of the wall is longitudinally extended, the diameter of the first lengthwise portion of the wall increasing from a reduced condition when the first lengthwise portion of the wall resiliently returns from a longitudinally extended condition.

2. (canceled)

3. A catheter assembly according to claim 1 wherein the opening in an exterior catheter surface at a distal end of the catheter is a first opening, the lumen extending throughout the length of the catheter and communicating with both the first opening and a second opening in an exterior catheter surface at a proximal end of the catheter.

4. A catheter assembly according to claim 1 wherein the catheter assembly includes only a single catheter and is free of any other catheter.

5. A catheter assembly according to claim 1 further comprising a stylet, the stylet having a length and an outer surface, the outer surface of the stylet including a portion oriented in a radial direction transverse to the length of the stylet, the inner surface of the wall of the catheter at least partially defining the lumen, the inner surface of the wall of the catheter including a portion oriented in a radial direction transverse to the length of the catheter, the portion of the outer surface of the stylet being positioned to transmit pressure to the portion of the inner surface of the wall of the catheter so that pressure applied to the stylet causes longitudinal extension of the first lengthwise portion of the wall.

6. A catheter assembly according to claim 5 wherein the stylet is hollow and has an open distal end and an open proximal end.

7. A catheter assembly according to claim 5 further comprising a tubular member secured to the inner surface of the wall of the catheter adjacent the portion of the inner surface to transmit pressure from the stylet to the wall of the catheter.
